(19) United States
(12) Patent Application Publication
(10) Pub. No.: US 2019/0183600 A1
(43) Pub. Date: Jun. 20, 2019

(54) DEVICE FOR ANCHORING AND IDENTIFYING MULTIPLE SUSPECTED LESIONS ANCHORING DEVICES

(71) Applicants: Laurent FUMEX, MADISON, CT (US); Thierry MASSEGLIA, LA GARDE (FR)

(72) Inventors: Laurent FUMEX, Madison, CT (US); Thierry MASSEGLIA, LA GARDE (FR)

(21) Appl. No.: 16/197,538

(22) Filed: Nov. 21, 2018

(30) Foreign Application Priority Data
Nov. 22, 2017 (FR) 17 61057

(51) Int. Cl. A61B 90/00

(52) U.S. Cl. CPC A61B 90/39; A61B 2090/3908; A61B 2090/3991; A61B 2090/3983; A61B 2090/3987; A61B 2090/3912

(57) ABSTRACT

The invention relates to an anchoring device for registering lesions or microcalcification in a biological tissue, comprising:
a guide wire comprising at least one graduated portion for determining the anchoring depth of the anchoring device,
at least two anchoring members for anchoring in a biological tissue connected to the guide wire, and
a connecting ring connecting the guide wire to the anchoring members.

The invention relates also to an anchoring system comprising such an anchoring device, a mandrel and a hollow positioning needle.

The present invention relates to a radiological and surgical device, and more particularly a device for anchoring and registering pulmonary nodules in order for them to be surgically removed, and a placement apparatus.

Lung cancer is a cancer affecting a large number of patients and reflecting the highest mortality rate. The use of biopsy for histological diagnosis has therefore become an essential procedure and all the more so given that the rate of malignancy in pulmonary nodules is very high. The biopsies are traditionally performed by transbronchial pathway or by percutaneous pathway using fine needles under a scanner. However, the increasingly early screening of the nodules and their geography can limit these traditional biopsy techniques making surgical intervention de facto necessary.

When surgery is possible, there are two techniques: thoracotomy and thoracoscopy.

Thoracotomy consists in making a surgical incision in the thoracic wall. The surgery may require the opening of the thorax or simply an incision between the sides. This so-called “open” surgery is invasive and difficult for the patient to withstand.

Thoracoscopy is a minimally invasive surgery of the thorax. It is a so-called “video-assisted” surgery involving making several small incisions (approximately 2 cm long) using long and fine instruments. The light and the vision are supplied by an optical device introduced through one of these incisions. This latter technique is therefore very advantageous but necessarily requires the nodule or nodules that have to be removed to be located and registered beforehand.

The registering of the nodules is thus entrusted, prior to the surgical act, to radiologists, who have recourse to preoperative registration techniques more often than not using a spear placed, under tomodensitometric control, in proximity to the nodules being removed.

Currently, most of the spears used in a pulmonary nodule registration procedure have been designed to register lesions and microcalcifications of the breast and are not suited for use in thoracoscopy. Indeed, to be put in place by the radiologist in total safety under tomodensitometric control and to provide reliable registration to the surgeon during the ablation, the registration system must first of all include a placement apparatus in which the outer diameter of the needle is suited to the surgical constraints, preferably less than 1.25 mm (18 gauge), and in particular in order to reduce the risks of pneumothorax; the system must then be able to adapt to the movements of amplitude induced by the inflation and the deflation of the lung and of the thoracic cage during the ventilation phases imposed by the thoracoscopy; this is in order to ensure the durability of the anchoring in the tissues throughout the procedure. Finally the device must offer an exceptional quality of anchoring in the pulmonary tissues whose very composition and texture make any catching difficult, and to do so in order to avoid any risk of migration of the device and therefore of loss of registration before and during the surgery.

An example of a known anchoring device is described in the document FR 2 731 343 A. This anchoring device comprises a sleeve of which one end is secured to a wire and the other, opposite end is secured to a strand of circular or rectangular section suitable for being anchored in the breast tissues to register a nodule. To secure the strand inside the sleeve, the strand is crimped in the sleeve so as to secure them to one another. The anchoring device can comprise several strands crimped inside the sleeve. In the latter case, the dimensions of the sleeve must be adapted to accommodate all of the strands. As indicated above, such an anchoring device is not however suited for use in thoracoscopy. Indeed, it has been found that by using such an anchoring device having dimensions suited to thoracoscopy, that is to say when it comprises only a single strand, its anchoring capacity is too weak such that the anchoring device has a tendency to migrate.

There is therefore a need for a device for anchoring and registering pulmonary nodules for the surgical ablation thereof having dimensions suited to thoracoscopy, also capable of absorbing the amplitudes associated with thoracoscopy and finally having an anchoring capacity suited to the structure of the pulmonary tissues making it possible to avoid the migration of the anchoring device before and during the surgery.

For that, the invention relates to an anchoring device for registering lesions or microcalcification in a biological tissue, comprising:
a guide wire,
at least two anchoring members for anchoring in a biological tissue connected to the guide wire, and
a connecting ring connecting the guide wire to the anchoring members, in which each anchoring member comprises a connecting end extending along a longitudinal axis and configured to be arranged inside the connecting ring, each connecting end having a transverse section forming an angular segment, the sum of the angular segments being equal to or less than 360° in one and the same plane transversal to the longitudinal axis.

This configuration of the anchoring device makes it possible to produce the connection of a plurality of anchoring members inside a connecting ring having dimensions similar to those used when the anchoring device comprises a single anchoring member. In other words, this configuration makes it possible to reduce the bulk of the anchoring members inside the connecting ring. Now, increasing the number of anchoring members makes it possible to improve the anchoring capacity of the device. Thus, the anchoring capacity of the anchoring device can be improved while having an anchoring device whose dimensions are conformal with thoracoscopy.

According to an embodiment of the anchoring device, the connecting ends are complementary to one another so as to form a circular section when the connecting ends are arranged in contact with one another.

According to an embodiment of the anchoring device, each connecting end comprises an outer surface in contact with the connecting ring and at least one inner surface arranged in contact with at least one other connecting end.

According to an embodiment of the anchoring device, the outer surface is curved and the inner surface is planar.

According to an embodiment of the anchoring device, the anchoring members comprise an anchoring portion formed in the extension of the connecting end, the anchoring portion comprising at least one planar surface extending transversely to the longitudinal axis.

According to an embodiment of the anchoring device, the anchoring members comprise, at an end opposite the connecting end, a leading edge for perforating the biological tissue, formed by at least one facet.

According to an embodiment of the anchoring device, each anchoring member is configured to be arranged in:
a first position in which the anchoring member has a configuration making it possible to insert the anchoring member into a biological tissue, and
a second position in which the anchoring member has a configuration making it possible to retain the anchoring member in a biological tissue.

According to an embodiment of the anchoring device, the anchoring members are configured to extend in different directions from one another when the anchoring members are each arranged in their second position.

According to an embodiment of the anchoring device, the anchoring members are each produced in an elastic material allowing the anchoring members to be deformed between the first and second positions.

According to an embodiment of the anchoring device, the latter further comprises an abutment forming a bearing surface for the longitudinal driving of the anchoring device.

According to an embodiment of the anchoring device, the guide wire comprises at least one graduated portion for determining the anchoring depth of the anchoring device.

According to an embodiment of the anchoring device, the graduated portion is planar.

According to an embodiment of the anchoring device, the graduated portion comprises a local widening of the transverse section of the wire forming the abutment.

According to an embodiment of the anchoring device, the graduated portion is arranged at a predetermined length from the connecting ring.

The invention relates also to an anchoring system for registering lesions or microcalcification in a biological tissue, comprising:
a hollow positioning needle,
an anchoring device according to any one of the preceding claims configured to slide inside the hollow positioning needle,
a mandrel configured to slide inside the hollow positioning needle so as to drive the anchoring members out of the hollow positioning needle.

According to an embodiment of the anchoring system, at least one out of the two anchoring members of the anchoring device comprises a transverse section equal to or less than half the inner diameter of the hollow positioning needle.

According to an embodiment of the anchoring system, the mandrel comprises a through-hole allowing the mandrel to slide around and along the guide wire of the anchoring device.

According to an embodiment of the anchoring system, the mandrel is configured to drive the anchoring members by bearing on the abutment.

According to an embodiment of the anchoring system, the hollow positioning needle is configured to arrange the anchoring members in the first position when the anchoring members are arranged inside the hollow positioning needle, the anchoring members being configured to be deformed in order to adopt the second position once driven out of the hollow positioning needle.

Other features and advantages of the invention will become apparent on reading the following description of preferred embodiments of the invention, given by way of example and with reference to the attached drawings.

FIG. 1 represents a perspective view of an anchoring system comprising a mandrel and an anchoring device arranged inside a hollow needle.

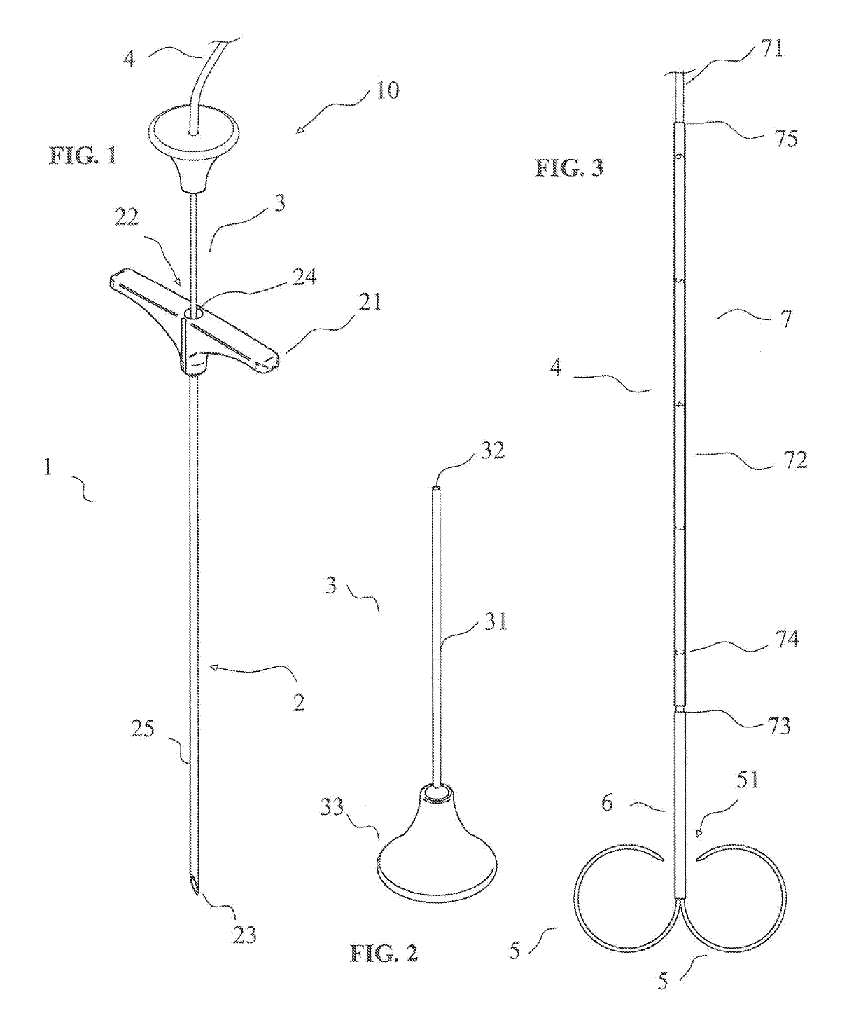

FIG. 2 represents a perspective view of the mandrel out of the hollow needle.

FIG. 3 represents a front view of the anchoring device of FIG. 1 out of the hollow needle.

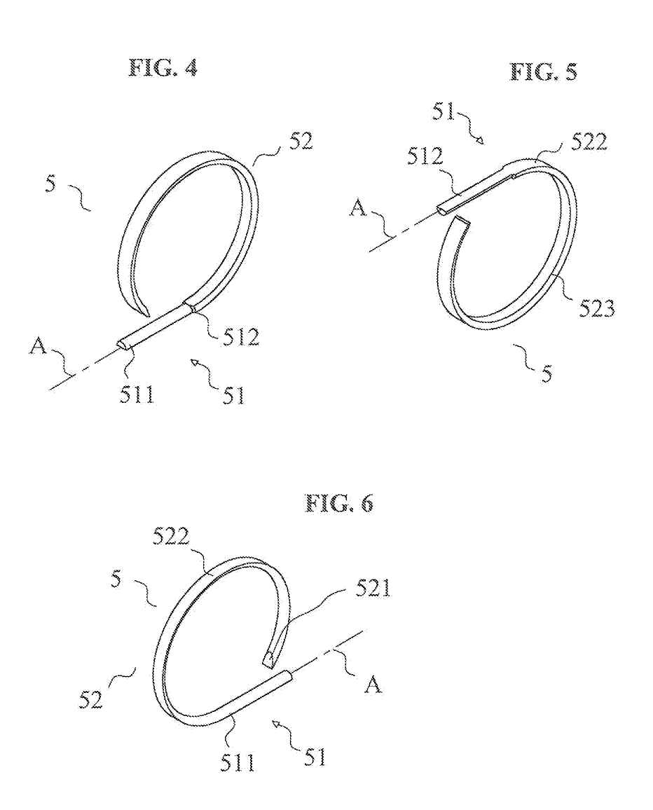

FIGS. 4 and 5 represent a perspective view of an embodiment of an anchoring member of the anchoring device of FIGS. 1 and 3.

FIG. 6 represents a perspective view of another embodiment of an anchoring member of the anchoring device of FIGS. 1 and 3.

As represented in FIG. 1, an anchoring system 10 for registering lesions or microcalcification in a biological tissue comprises a hollow positioning needle 2 and an anchoring device 4 configured to slide inside the hollow positioning needle 2. The anchoring system 10 also comprises a mandrel 3 configured to slide inside the hollow positioning needle 2 so as to drive the displacement of the anchoring device 4 out of the hollow positioning needle 2. The displacement of the anchoring device 4 by the mandrel 3 is done by sliding along the hollow positioning needle 2.

To allow the sliding of the mandrel 3 and of the anchoring device 4 inside the hollow positioning needle 2, the latter forms a longitudinally-extending through-hole 22. The hollow positioning needle 2 comprises a perforation end 23 configured to perforate one or more types of tissues. In particular, the perforation end 23 is bevelled so as to form a point capable of perforating the tissues. The perforation end 23 constitutes an end for the anchoring device 4 to enter when it is mounted in the hollow positioning needle 2 and for the anchoring device 4 to exit from the hollow positioning needle 2 when it is driven by the mandrel 3. The hollow positioning needle 2 also comprises an end 24 for insertion of the mandrel 3. The insertion end 24 is opposite the perforation end 23. The through-hole 22 extends between the perforation 23 and insertion 24 ends. The hollow positioning needle 2 extends preferably over a length between 100 mm and 150 mm. The inner diameter of the hollow positioning needle 2 is preferably equal to or less than 0.95 mm. For the manipulation of the anchoring system 10, the hollow positioning needle 2 also comprises a handle 21 arranged at the insertion end 24. The handle 21 is configured to receive, on either side of the handle, two fingers of a user to produce a force for retaining the hollow positioning needle 2 when the mandrel 3 is pushed inside the hollow positioning needle 2. Preferably, the user places his index and middle fingers under the handle 21 so as to actuate the mandrel 3 with his or her thumb, like a syringe. The handle 21 is for example overmoulded around a needle body 25 forming the through-hole 22. Preferably, the handle 21 is made of plastic material and the needle body 25 is made of metal material.

The needle body 25 is preferably of circular section. Alternatively, the needle body 25 can be of any section, for example rectangular, square or oval.

As represented in FIG. 2, the mandrel 3 comprises a mandrel body 31 and a plunger 33 mounted on the mandrel body 31. The plunger 33 is preferably overmoulded on the mandrel body 31. More preferably, the mandrel body 31 is produced in a metal material and the plunger 33 is produced in a plastic material. The plunger 33 is configured such that the finger of a user can act on a distal portion of the plunger 33 so as to drive the anchoring device 4. Thus, in combination with the handle 21, the anchoring system 10 can be used with the use of only three fingers, preferably the middle finger, the index finger and the thumb, like a syringe. The anchoring system 10 is thus simple to use because it is configured to be used with the use of only one hand.

The mandrel 3 also comprises a driving end 32 capable of cooperating with an abutment of the anchoring device 4 to drive the sliding of the anchoring device 4 inside the hollow positioning needle 2.

As represented in FIG. 3, the anchoring device 4 also comprises a guide wire 7 and two anchoring members 5 for anchoring in a biological tissue connected to the guide wire 7. The two anchoring members 5 are assembled at one end of the guide wire 7 using a connecting ring 6. In particular, an end of the guide wire 7 is crimped inside an end of the connecting ring 6. Likewise, an end of each of the anchoring members 5 is crimped inside the ring at an end of the connecting ring 6 opposite the guide wire 7. Thus, the guide wire 7, the connecting ring 6 and the anchoring members 5 form an integral assembly.

This integral assembly formed by the guide wire 7, the connecting ring 6 and the anchoring members 5 is intended to be inserted inside the hollow positioning needle 2 and the mandrel 3 to be introduced into the tissues of a patient. Alternatively, the guide wire 7, the connecting ring 6 and the anchoring members 5 can be assembled together by any means allowing this integral assembly to be inserted into the mandrel 3.

The guide wire 7 comprises at least three portions, a proximal portion 73 intended to be crimped in the connecting ring 6, the graduated portion 72 configured for centimetric marking and whose outer diameter is greater than the outer diameter of the proximal portion 73 and greater than the inner diameter of the body of the mandrel 31, and a distal portion 71 configured to slide in the body of the mandrel 31. The guide wire 7 preferably corresponds to a flexible wire whose intermediate portion between its ends is flattened to form the graduated portion 72. “Flexible” is understood to mean the fact that the wire can be wound on itself so as to form a loop of a diameter less than 10 cm, preferably less than 6 cm, without plastic deformation of the wire. As an example, the flexible wire can be made of polymer material, such as biocompatible polyamide. The wire can also be metal and braided, i.e. composed of several strands interwoven with one another. Of the three portions of the guide wire 7, at least the graduated portion 72 of the guide wire 7 is flexible. As a comparison, a “rigid” wire corresponds to a solid metal wire of the same outer diameter.

The guide wire 7 comprises at least one graduated portion 72 for determining the anchoring depth of the anchoring device 4. For that, said graduated portion comprises graduations 74, for example centimetric, formed on the graduated portion 72. Furthermore, the graduated portion 72 is arranged at a predetermined length from the connecting ring 6. When the anchoring device 4 is arranged inside the biological tissues, the guide wire 7 extends out of the incision made for the insertion of the anchoring device 4. In particular, the guide wire 7 is configured such that the graduated portion 72 extends out of this incision such that the surgeon can know the anchoring depth of the anchoring device 4. Preferably, the graduated portion 72 is planar or comprises a planar graduated surface to facilitate the production of the graduation 74. The graduated portion 72 is preferably produced by a localized flattening of the guide wire 7 to obtain a planar graduated portion 72. The guide wire 7 can comprise a plurality of graduated portions 72 to form a discontinuous graduation zone. In the latter case, each graduated portion 72 can comprise one or more graduations 74 while retaining a predefined distance between each graduation 74.

The guide wire 7 also comprises an abutment 75 forming a bearing surface for the longitudinal driving of the anchoring device 4. The abutment 75 forms a protuberance transversal to the direction of extension of the guide wire 7. When the anchoring device 4 is arranged inside the hollow positioning needle 2 with the mandrel 3 mounted on the distal portion 71 of the guide wire 7, this protuberance is configured to form a longitudinal bearing abutment for the driving end 32 so as to drive the anchoring device 4 inside the hollow positioning needle 2. Thus, the mandrel 3 is preferably arranged around the guide wire 7 in an upper part of the guide wire 7 arranged above the abutment 75.

The abutment 75 is preferably produced by the graduated portion 72. In effect, the graduated portion 72 can comprise a local widening of the transverse section of the guide wire 7 forming the abutment 75. The abutment 75 is preferably produced by an end of the graduated portion 72. When the guide wire 7 is flattened, a local increase in a transverse dimension of the guide wire 7 is generated to form the abutment 75. To allow the abutment 75 to perform its function of bearing abutment, this dimension transversal to the direction of extension of the wire is greater than the inner diameter of the mandrel body 31. For the sliding of the anchoring device 4 inside the hollow positioning needle 2, this transverse dimension is also less than the inner diameter of the needle body 25.

The guide wire 7 is produced in a flexible material used for suturing threads, preferably made of biocompatible polyamide. Thus, the guide wire 7 makes it possible not to injure the patient while he or she is breathing and to exert no force on the anchoring device 4 and therefore reduce the risks of migration. The production of a graduated portion 72 in the material of the guide wire 7 makes it possible to conserve its advantages while providing information to the surgeon on the anchoring depth.

The dimensions of the mandrel 3 are configured to allow the mandrel 3 to be arranged inside the hollow positioning needle 2, between the anchoring device 4 and the hollow positioning needle 2. For that, the outer diameter of the mandrel body 31 is less than the inner diameter of the needle body 25 and the inner diameter of the mandrel body 31 is greater than the outer diameter of the distal portion 71 of the guide wire 7 of the anchoring device 4 while being less than the transverse section of the abutment 75 of the guide wire 7. The mandrel body 31 is preferably of circular section. Alternatively, the mandrel body 31 can be of any section allowing the mandrel 3 to slide inside the hollow positioning needle 2 and, at the distal portion 71 of the guide wire 7 of the anchoring device 4, to slide inside the mandrel 3.

Each anchoring member 5 is configured to be arranged in a first or a second position. In the first position, the anchoring member 5 has a configuration making it possible to insert the anchoring member 5 into a biological tissue. In other words, the anchoring member 5 is configured to be arranged in an extended position allowing it to be inserted into the hollow positioning needle 2. The hollow positioning needle 2 can therefore be inserted into the biological tissues of the patient with the anchoring member 5 arranged within it. In this first position, the anchoring member 5 does not itself perforate the biological tissues of the patient. The first position can thus be qualified as inactive or as position of non-retention of the biological tissues. In the second position, the anchoring member 5 has a configuration making it possible to retain the anchoring member 5 in a biological tissue. In other words, the anchoring member 5 is configured to be arranged in a retention position capable of being set in opposition relative to biological tissues in which it is inserted. In this retention position, the anchoring member 5 can take the form of a hook. The second position can therefore be qualified as active or position of retention of the biological tissues of the patient. Consequently, each anchoring member 5 is configured to be arranged in extended position inside the hollow positioning needle 2 before anchoring and in retention position when the anchoring device 4 is driven by the mandrel 3 inside the hollow positioning needle 2. Thus, when the hollow positioning needle 2 is inserted into the body of a patient, the actuation of the mandrel 3 makes it possible to deploy the anchoring members 5 out of the hollow positioning needle 2 to anchor them in the biological tissues. In other words, the hollow positioning needle 2 is configured to arrange the anchoring members 5 in the first position when the anchoring members 5 are arranged inside the hollow positioning needle 2, the anchoring members 5 being configured to be deformed in order to adopt the second position once driven out of the hollow positioning needle 2.

According to a preferred configuration, the anchoring member 5 comprises a free end configured to perforate the biological tissues. In other words, the anchoring member 5 has an open profile with a proximal end secured to the guide wire 7 and a distal end for perforation of the biological tissues. Thus, when the anchoring member 5 is moved from the first position to the second position, the free distal end allows the anchoring member 5 to perforate the biological tissues and to be held therein once in the second position. This configuration with a free end allows the anchoring member 5 to be anchored in a portion of biological tissue without having to deform it or pinch it.

To enhance the anchoring of the anchoring members 5, the latter can be configured to extend in directions different from one another when the anchoring members 5 are each arranged in their second position. The anchoring members 5 are preferably each produced in an elastic material allowing the anchoring members 5 to be deformed between the first and second positions. More preferably, the anchoring members 5 are produced in polymer with shape memory, in organic or metal alloy with shape memory, in polyethylene or in a nickel/titanium alloy.

Each anchoring member 5 comprises a connecting end 51 extending along a longitudinal axis A and configured to be arranged inside the connecting ring 6. When the connecting ends 51 are arranged inside the connecting ring 6 and the anchoring device is arranged inside the hollow positioning needle 2, the longitudinal axis A coincides with the axis of extension of the hollow positioning needle 2. Furthermore, each connecting end 51 has a transverse section forming an angular segment, the sum of the angular segments being equal or less than 360° in one and the same plane transversal to the longitudinal axis A. In other words, each connecting end 51 occupies only a portion of the inner space of the connecting ring 6, in a plane transversal to the longitudinal axis A. Thus, it is possible to occupy a maximum of space inside the connecting ring 6 such that the securing of each of the anchoring members 5 can be accomplished efficiently. This configuration makes it possible to reduce the bulk of the anchoring members 5 inside the connecting ring 6. This is particularly useful because the radial dimensions of the connecting ring 6 are constrained by the use of the anchoring device 4 in thoracoscopy. Thus, it is possible to improve the fixing of a plurality of anchoring members 5 inside the connecting ring 6.

Furthermore, the connecting ends 51 are preferably complementary to one another so as to form a circular section when the connecting ends 51 are arranged in contact with one another. Preferably, the circular section formed by the connecting ends 51 is smaller than the inner diameter of the connecting ring 6. As an example, when the anchoring device 4 comprises two anchoring members 5, the anchoring members 5 comprise a connecting end 51 of semicircular section. As another example, when the anchoring device 4 comprises three anchoring members 5, the anchoring members 5 comprise a connecting end 51 whose section forms an angular segment of 120°. Each connecting end 51 comprises an outer surface 511 in contact with the connecting ring 6 and at least one inner surface 512 arranged in contact with at least one other connecting end 51. The outer surface 511 is preferably curved and the inner surface 512 is preferably planar. The outer or inner nature of the outer 511 and inner 512 surfaces is defined by considering the anchoring member 5 when it is arranged inside the hollow positioning needle 2. To improve the mutual grip of the anchoring members 5 inside the connecting ring 6, the inner surface 512 can include a relief, provided for example by a surface roughness, grooves, ridges or notches. Preferably, each anchoring member 5 comprises an identical connecting end 51. Alternatively, each connecting end 51 can be different, in particular by forming an angular segment of different value. As an example, with two anchoring members 5, one of the connecting ends 51 can exhibit an angular segment less than 180° and the other of the connecting ends 51 can exhibit an angular segment greater than 180°. The anchoring members 5 are preferably assembled in the connecting ring 6 by crimping, gluing, welding or by collapsing of the connecting ring 5 on the connecting ends 51.

The anchoring member 5 also comprises an anchoring portion 52 formed in the extension of the connecting end 51. The anchoring portion 52 comprises at least one planar surface extending transversely to the longitudinal axis A to improve the capacity of the anchoring member 5 to be retained in the biological tissues. Thus, this planar surface allows an effective anchoring of the anchoring member 5 in the tissues such that the risks of a migration of the anchoring device 4 is reduced. The anchoring portion 52 also comprises an outer surface 522 and an inner surface 523. The outer or inner nature of the inner 523 and outer 522 surfaces is defined by considering the anchoring member 5 when it is out of the hollow positioning needle 2. Preferably, the planar surface is formed by the outer surface 522. More preferably, the outer surface 522 is produced in the extension of the inner surface 512 of the connecting end 51. This anchoring portion 52 is preferably curved when the anchoring member 5 is in the second position so as to form a hook and thus improve the anchoring of the anchoring member 5. According to the embodiment represented in FIGS. 4 and 5, at least one of the anchoring members 5 has a transverse section forming a parallelogram, preferably a rectangle. For example, the anchoring portion 52 can be obtained from a flattened semicircular form. The thickness of the anchoring portion 52 in a direction transversal to the longitudinal axis A is chosen as a function of the grip on deployment that is required to be given to the anchoring portion 52. When the anchoring device 4 comprises two anchoring members 5, this thickness is equal to or less than half the inner diameter of the hollow positioning needle 2. All the forms contained in this half-diameter are allowed. According to a preferential embodiment, the deployed form of the anchoring member 5 is produced by stamping a cylindrical axis whose diameter is previously defined in order to obtain the different sections.

The anchoring members 5 also comprise, at an end opposite the connecting end 51, a leading edge 521 for perforating the biological tissue, formed by at least one facet or bevel. This facet or bevel is for example formed by the anchoring portion 52. The main facet or bevel of this tapering is produced on the inner surface of the anchoring member 5 so as to favour the slip of the anchoring member 5 when it is ejected from the hollow positioning needle 2. This leading edge 521 also allows the anchoring embers 5 to perforate the biological tissues to place the anchoring device 4 in anchoring position.

According to FIG. 6, the anchoring members 5 can be produced according to another embodiment in which the anchoring portion 52 has a semicircular transverse section. Preferably, the anchoring members 5 have a continuous semicircular section. In other words, the connecting end 51 and the anchoring portion 52 can have a semicircular transverse section. In this way, when the anchoring device 4 comprises two anchoring members 5, the latter can be formed by a rod of semicircular section whose end forms the connecting end 51, the rest being formed by the anchoring portion 52. The embodiment of FIG. 6 makes it possible to obtain an economical anchoring member 5 obtained with few manufacturing operations while guaranteeing a good retention of the anchoring member 5 in the biological tissues with a planar outer surface 522. The anchoring member according to the embodiment of FIG. 6 differs from the embodiment of FIGS. 4 and 5 in that the transverse section of the anchoring portion 52 is semicircular and produced in the extension of the connecting end 51. However, all of the other features described for the anchoring member 5 of FIGS. 4 and 5 are compatible with the embodiment of FIG. 6. Obviously, when the anchoring device 4 comprises three anchoring members 5 according to the embodiment of FIG. 6, the transverse section of the anchoring portion 52 and of the connecting end 51 form an angular portion corresponding to a third of a circle.

As illustrated in FIGS. 4 to 6, the end opposite the connecting end 51 forms a free end, i.e. it is not linked or secured to the connecting end 51. This end opposite the connecting end 51 is configured to perforate biological tissues, in particular by the presence of the leading edge 521.

Also proposed is a first alternative anchoring device according to the following items:
Item 1: Anchoring device for registering lesions or microcalcification in a biological tissue, comprising:
   a guide wire comprising at least one graduated portion for determining the anchoring depth of the anchoring device,
   at least two anchoring members for anchoring in a biological tissue connected to the guide wire.
Item 2: Anchoring device according to item 1, in which the graduated portion is planar.
Item 3: Anchoring device according to item 1 or 2, in which the graduated portion is obtained by flattening of the guide wire.
Item 4: Anchoring device according to any one of the preceding items, also comprising an abutment forming a bearing surface for the longitudinal driving of the anchoring device.
Item 5: Anchoring device according to item 4, in which the graduated portion comprises a local widening of the transverse section of the wire forming the abutment.
Item 6: Anchoring device according to one of the preceding items, comprising a connecting ring for connecting the guide wire to the anchoring members, in which the graduated portion is arranged at a predetermined length from the connecting ring.
Item 7: Anchoring device according to one of the preceding items, in which the guide wire is produced in a flexible material, preferably in biocompatible polyamide.

Moreover, also proposed is a second alternative anchoring device according to the following items:
Item 1: Anchoring device for registering lesions or microcalcification in a biological tissue, comprising:
   a guide wire,
   at least two anchoring members for anchoring in a biological tissue connected to the guide wire, each anchoring member comprising a connecting end extending along a longitudinal axis, the anchoring members comprising a planar retaining surface extending transversely to the longitudinal axis.
Item 2: Anchoring device according to item 1, in which at least one of the anchoring members has a transverse section forming a parallelogram, preferably a rectangle.
Item 3: Anchoring device according to item 1 or 2, in which the anchoring members comprise, at an end opposite the connecting end, a leading edge for perforating the biological tissue, formed by at least two facets.

Moreover, also proposed is a third alternative anchoring device according to the following items:
Item 1: Anchoring device for registering lesions or microcalcification in a biological tissue, comprising:
   a guide wire,
   at least two anchoring members for anchoring in a biological tissue connected to the guide wire, and
   a connecting ring connecting the guide wire to the anchoring members, in which each anchoring member comprises a connecting end extending along a longitudinal axis and configured to be arranged inside the connecting ring, each connecting end having a transverse section forming an angular segment, the sum of the angular segments being equal to or less than 360° in one and the same plane transversal to the longitudinal axis.
Item 2: Anchoring device (4) according to item 1, in which the connecting ends (51) are complementary to one another so as to form a circular section when the connecting ends (51) are arranged in contact with one another.
Item 3: Anchoring device (4) according to item 1 or 2, in which each connecting end (51) comprises an outer surface (511) in contact with the connecting ring (6) and at least one inner surface (512) arranged in contact with at least one other connecting end (51).
Item 4: Anchoring device (4) according to item 3, in which the outer surface (511) is curved and the inner surface (512) is planar.
Item 5: Anchoring device (4) according to any one of the preceding items, in which the anchoring members (5) comprise an anchoring portion (52) formed in the extension of the connecting end (51), the anchoring portion (52) comprising at least one planar surface extending transversely to the longitudinal axis (A).
Item 6: Anchoring device (4) according to any one of the preceding items, in which the anchoring members (5) comprise, at an end opposite the connecting end (51), a leading edge (521) for perforating the biological tissue, formed by at least one facet.
Item 7: Anchoring device (4) according to any one of the preceding items, in which each anchoring member (5) is configured to be arranged in:
   a first position in which the anchoring member (5) has a configuration making it possible to insert the anchoring member (5) into a biological tissue, and
   a second position in which the anchoring member (5) has a configuration making it possible to retain the anchoring member (5) in a biological tissue.
Item 8: Anchoring device (4) according to item 7, in which the anchoring members (5) are configured to extend in directions that are different from one another when the anchoring members (5) are each arranged in their second position.

The first, second and third alternative anchoring devices can also comprise one or more of the features described in the present description taken in any technically admissible combination.

It should be noted that each of the alternative anchoring devices presented according to the preceding items is configured to be implemented in an anchoring system as described hereinabove and compatible with all of the features of the anchoring device 4 presented hereinabove.

1. Anchoring device for registering lesions or microcalcification in a biological tissue, comprising:
a guide wire comprising at least one graduated portion for determining the anchoring depth of the anchoring device,
at least two anchoring members for anchoring in a biological tissue connected to the guide wire, and
a connecting ring connecting the guide wire to the anchoring members.

2. Anchoring device according to claim 1, in which the graduated portion is planar.

3. Anchoring device according to claim 1, in which the guide wire comprises an abutment forming a bearing surface for the longitudinal driving of the anchoring device by a mandrel.

4. Anchoring device according to claim 3, in which the graduated portion comprises a local widening of the transverse section of the wire forming the abutment.

5. Anchoring device according to claim 1, in which the graduated portion is arranged at a predetermined length from the connecting ring.

6. Anchoring device according to claim 1, in which the guide wire is produced in a flexible material, preferably in biocompatible polyamide.

7. Anchoring device according to claim 1, in which the guide wire further comprises:
a proximal portion intended to be crimped in the connecting ring, the outer diameter of the proximal portion being smaller than a maximum transverse dimension of the graduated portion, and
a distal portion configured to slide in the body of a mandrel.

8. Anchoring device according to claim 1, in which each anchoring member comprises a connecting end extending along a longitudinal axis and configured to be arranged inside the connecting ring, each connecting end having a transverse section forming an angular segment, the sum of the angular segments being equal to or less than 360° in one and the same plane transversal to the longitudinal axis.

9. Anchoring device according to claim 8, in which the connecting ends are complementary to one another so as to form a circular section when the connecting ends are arranged in contact with one another.

10. Anchoring device according to claim 8, in which each connecting end comprises an outer surface in contact with the connecting ring and at least one inner surface arranged in contact with at least one other connecting end.

11. Anchoring device according to claim 10, in which the outer surface is curved and the inner surface is planar.

12. Anchoring device according to claim 1, in which the anchoring members comprise an anchoring portion formed in the extension of the connecting end, the anchoring portion comprising at least one planar surface extending transversely to the longitudinal axis.

13. Anchoring device according to claim 12, in which the anchoring portion comprises a planar outer surface and a curved inner surface.

14. Anchoring device according to claim 1, in which the anchoring members comprise, at an end opposite the connecting end, a leading edge for perforating the biological tissue, formed by at least one facet.

15. Anchoring device according to claim 1, in which each anchoring member is configured to be arranged in:
a first position in which the anchoring member has a configuration making it possible to insert the anchoring member into a biological tissue, and
a second position in which the anchoring member has a configuration making it possible to retain the anchoring member in a biological tissue.

16. Anchoring device according to claim 15, in which the anchoring members are each produced in an elastic material allowing the anchoring members to be deformed between the first and second positions.

17. Anchoring system for registering lesions or microcalcification in a biological tissue, comprising:
a hollow positioning needle,
an anchoring device configured to slide inside the hollow positioning needle, the anchoring device comprising a guide wire with at least one graduated portion for determining the anchoring depth of the anchoring device, at least two anchoring members for anchoring in a biological tissue connected to the guide wire, and a connecting ring connecting the guide wire to the anchoring members,
a mandrel configured to slide inside the hollow positioning needle so as to drive the anchoring members out of the hollow positioning needle.

18. Anchoring system according to claim 17, in which at least one out of the two anchoring members of the anchoring device comprises a transverse section equal to or less than half the inner diameter of the hollow positioning needle.

19. Anchoring system according to claim 17, in which the mandrel comprises a through-hole allowing the mandrel to slide around and along the guide wire of the anchoring device.

20. Anchoring system according to claim 17, in which each anchoring member is configured to be arranged in a first position in which the anchoring member has a configuration making it possible to insert the anchoring member into a biological tissue, and
a second position in which the anchoring member has a configuration making it possible to retain the anchoring member in a biological tissue, the hollow positioning needle being configured to arrange the anchoring members in the first position when the anchoring members are arranged inside the hollow positioning needle, the anchoring members being configured to be deformed in order to adopt the second position once driven out of the hollow positioning needle.
